(19) United States
(12) Patent Application Publication
(10) Pub. No.: US 2012/0253315 A1
(43) Pub. Date: Oct. 4, 2012

(54) INJECTION NEEDLE FOR FRACTURES

(75) Inventor: William F. McKay, Memphis, TN (US)

(73) Assignee: WARSAW ORTHOPEDIC, INC., Warsaw, IN (US)

(21) Appl. No.: 13/076,672

(22) Filed: Mar. 31, 2011

(51) Int. Cl. A61M 5/00

(52) U.S. Cl. 604/506; 604/272

(57) ABSTRACT

A cannula having proximal and distal ends defines a hollow body. A tissue-piercing tip is formed at the distal end of the cannula and is fluidically sealed from the hollow body. At least one opening is formed in a sidewall of the cannula and fluidically coupled to the hollow body. A fracture may be treated by advancing the tip through soft tissue so that the at least one opening is at least partially disposed within or adjacent to the fracture. The needle is oriented so that the at least one opening faces an exposed end of fracture or within the plane of the fracture line. Then, a therapeutic composition, such as a bone growth factor, is pressured through the hollow body to exit from the at least one opening.

### FIELD OF THE INVENTION

The present invention relates generally to needles. More particularly, the present invention discloses a needle that is used to deliver therapeutic compositions to a bone fracture.

### BACKGROUND OF THE INVENTION

Needles are often used when treating closed fractures to deliver therapeutic compositions to such fractures, and in particular to deliver therapeutic compositions comprising bone growth factors. See, for example, U.S. application Ser. No. 11/504,363, entitled “Flowable Carrier Matrix And Methods For Delivering to a Patient,” filed on Aug. 14, 2006, the contents of which are incorporated herein by reference. In such cases, the needle design may be conventional, and as shown in FIG. 1 comprises a cannula 2 with an opened, sharpened tip 4. The therapeutic compositions 8 are delivered from an opening 6 in the tip 4 of the needle.

Use of such conventional needles to deliver therapeutic compositions 8 to a closed fracture 1 requires X-ray or fluoroscopic imaging to ensure that the needle tip 4 is in the desired position before beginning the injection of the composition 8. The therapeutic composition 8 is then expelled in a forward direction, as shown in FIG. 1, which tends to make its delivered location uncontrollable. The cannula 2 is then retracted as the injection is continued, requiring repeated X-rays or fluoroscopic imaging to observe where the therapeutic composition 8 is going. Alternatively, if the needle is held stationary while expelling syringe, its contents are delivered in one location and not uniformly across the fracture site, affecting clinical efficacy.

It would therefore be desirable to have a device and related method for delivering therapeutic compositions to closed fractures that would not require repeated X-rays or fluoroscopic imaging, and which would provide better control of the delivery of the composition to the fracture.

### SUMMARY OF THE INVENTION

In one aspect a needle for delivering a therapeutic composition is disclosed. Various embodiment needles comprise a cannula that defines a hollow body. This hollow body runs along an axial axis of the cannula, and the cannula has a proximal end and a distal end. A tissue-piercing tip is formed at the distal end of the cannula and is fluidically sealed from the hollow body. At least one opening is formed in a sidewall of the needle, which opening is fluidically coupled to the hollow body. In a specific embodiment at least two openings are respectively disposed in opposing sidewalls of the cannula. In preferred embodiments the axial extent of the at least one opening covers at least 10% of a bone fracture line. In other embodiments a plurality of openings may be, for example, disposed in four sides of the cannula sidewall, or periodically or randomly distributed around the circumference of the cannula sidewall so that the therapeutic agent is uniformly distributed within the fracture line, thereby improving clinical efficacy.

In another aspect a method for treating a fracture is disclosed in which an embodiment needle is used. The tip is advanced through soft tissue so that the at least one opening is at least partially disposed within or adjacent to the fracture. The needle is oriented so that the at least one opening faces an exposed end of fracture or is within the fracture plane. Then, a therapeutic composition is caused to travel through the hollow body and exit through the at least one opening. In a preferred embodiment the therapeutic composition comprises a bone growth factor.

### BRIEF DESCRIPTION OF THE DRAWINGS

FIG. 1 illustrates delivery of a therapeutic composition to a closed fracture according to the prior art.

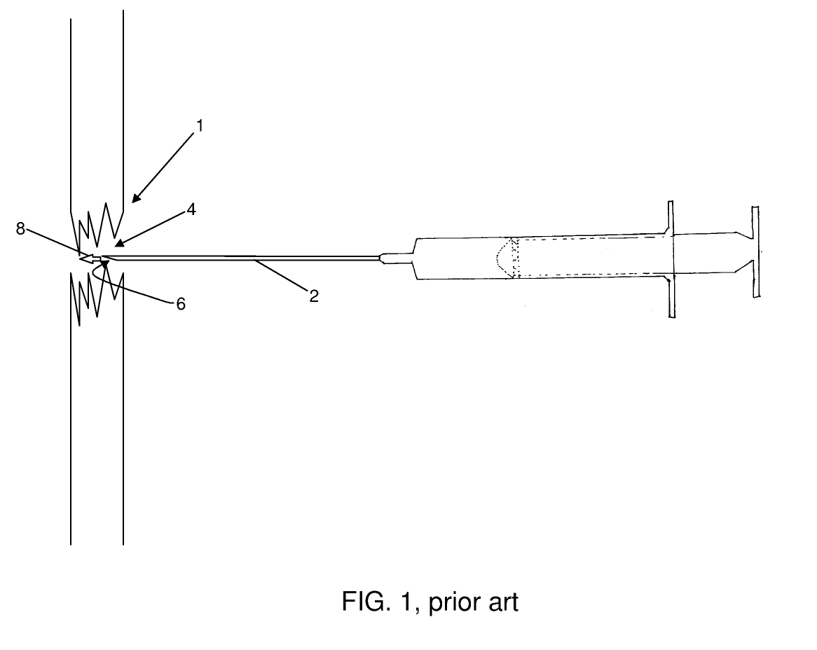

FIG. 2 shows a first embodiment needle.

FIG. 3 is a side view of a second embodiment needle.

FIG. 4 shows a third embodiment needle.

FIG. 5 is a side view of a fourth embodiment needle.

FIG. 6 illustrates delivery of a therapeutic composition to a closed fracture according to one embodiment of the present invention.

### DETAILED DESCRIPTION

Various embodiments disclose novel needle designs and related methods for treating closed fractures so that delivered therapeutic compositions, such as bone growth factors, are directed out the sides of the needle and into the ends of the fractured bones or plane of the fracture rather than out of the needle tip. This eliminates the need to repeatedly observe the needle tip via X-ray or fluoroscopic imaging as the needle is retracted. Embodiment devices and related methods also allow for the uniform distribution of the therapeutic agent within the fracture line between the ends of the exposed fractured bones, thereby improving clinical efficacy.

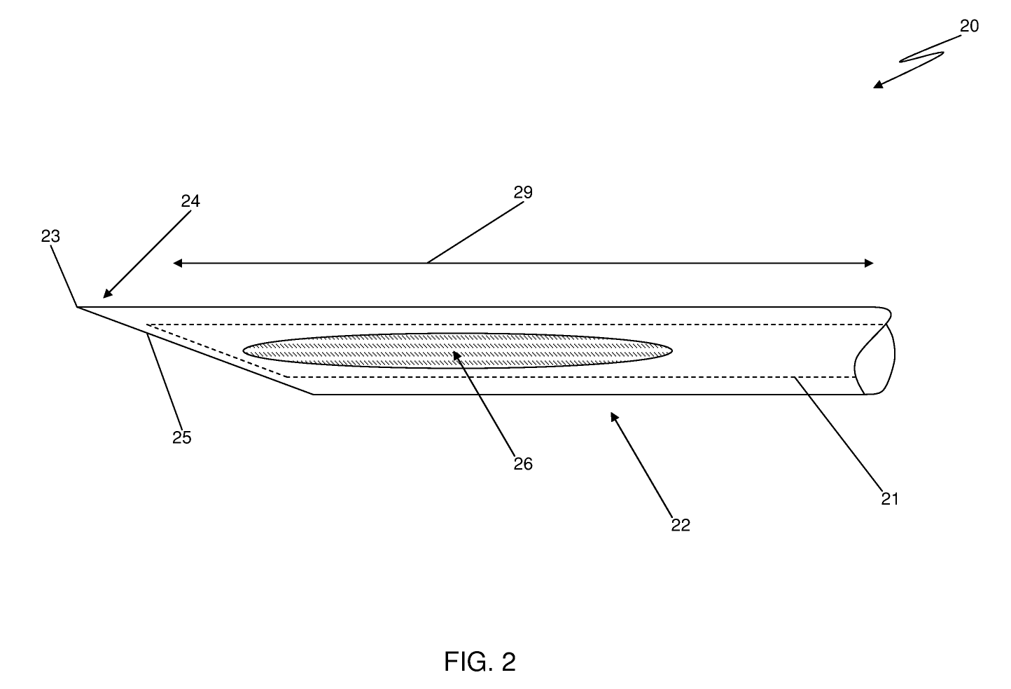

An embodiment needle 20 is shown in FIG. 2. The needle 20 includes a cannula 22 forming a hollow body 21 with a closed, sharpened tip 24 formed at a distal end of the cannula 22. The hollow body 21 extends along the axial axis 29 of the cannula 22, from the proximal end of the cannula 22 towards the distal end of the cannula 22. The proximal end of the cannula 22 (not shown in FIG. 2) may include, for example, any suitable locking mechanism for connecting the needle 20 to a syringe or the like. The cannula 22 can be made from any suitable material, but is preferably made from stainless steel. The cannula 22 can be made using standard hollow tube manufacturing methods. By way of example, the tip 24 can be plugged with a solid metal cylinder and welded in place or, a flat cover can be placed over the opening at the distal end of the cannula 22 and welded in place. Any suitable means for plugging the tip 24 may be employed, however. The tip 24 can be shaped as a standard hypodermic needle, having a tissue-piercing tip 23 and, optionally, cutting edges 25, as known in the art. However, the tip 24 is solid, and thus closed against the hollow body 21. That is, the tissue-piercing tip 24 fluidically seals the distal end of the cannula 22, and is thus sealed against the therapeutic composition traveling within the hollow body 21. Instead, one or more openings 26 in the cannula 22 are provided on the sidewall of the cannula 22 to provide fluidic communication between the space external to the cannula 22 and the hollow body 21 running axially down the middle of the cannula 22. These side openings 22 can be formed, for example, by either drilling and milling in desired slot shapes and sizes or via other metal cutting techniques as known in the art. Hence, the therapeutic compound traveling axially down the hollow body 21 does not exit the cannula 22 from the tip 24, but instead exits perpendicularly to the axial axis 29 of the cannula 22 from the one or more sidewall openings 26. In a preferred embodiment the axial extent of the one or more openings 26 (i.e., how long the opening(s) 26 extend along the cannula 22 in the axial direction 29) is great enough to substantially extend across the entirety of a bone fracture line. The cannula openings 26 can range, for example, from 25-1250 of the bone fracture line, with a preferred range of 50-1000. Various embodiment needles 20 can therefore be offered depending on the specific fractured bone to accommodate the varying bone diameter sizes, which typically range from 0.5-5.0 cm in diameter.

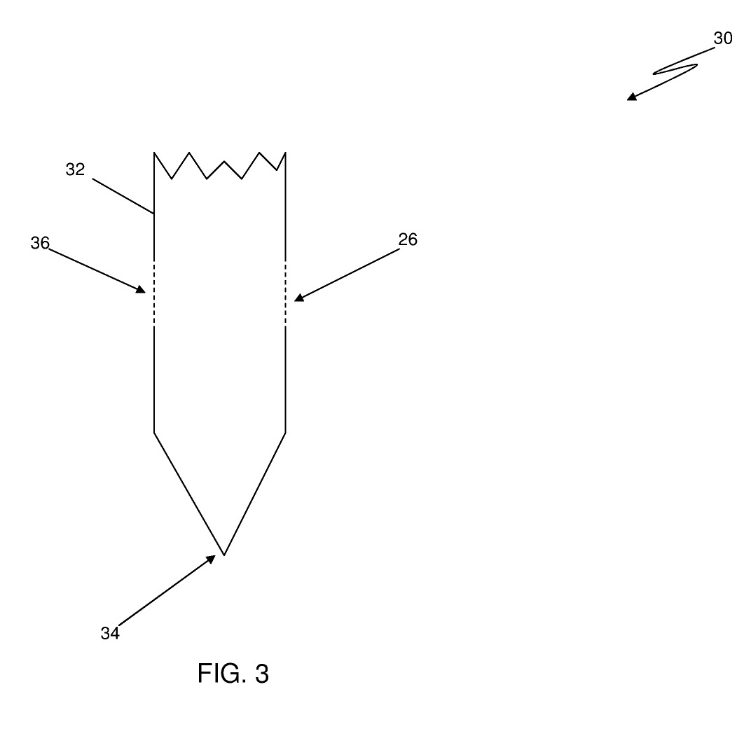

As shown in FIG. 2, in some embodiments only a single large opening 26 can be provided in the sidewall of the cannula 22. For purposes of the following, “large” can be defined as at least ⅓ of the cannula 22 circumference. This large opening 26 can start, for example, 2-10 mm from the tip 24 depending on the bone being treated. In another embodiment 30, as shown in FIG. 3, a cannula 32 can have, for example, two large openings 36 disposed in opposite sidewalls of the cannula 32. That is, a single opening 36 is disposed on each of two sidewalls, and the openings 36 in the sidewalls of the cannula can be displaced 180° from each other. These opposed openings 36 can be disposed at equal distances from the tip 34, or can be staggered so that one of the openings 36 is farther from the tip 34 than the other opening 36, as measured form the geometric centers of the respective openings 36. Staggering the openings 36 may provide more uniform distribution of the therapeutic agent, in particular for viscous agents.

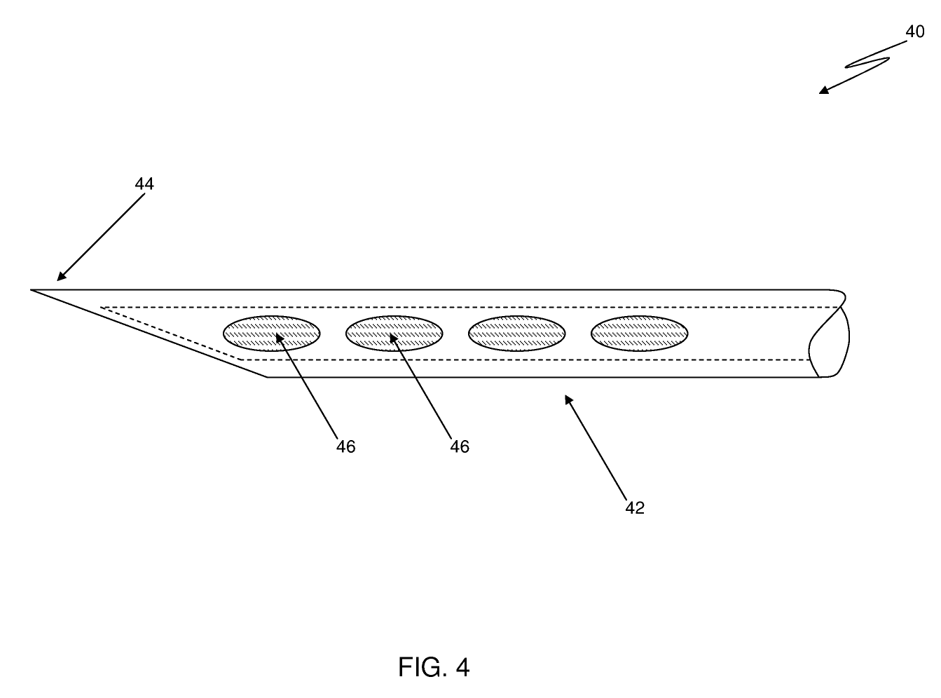

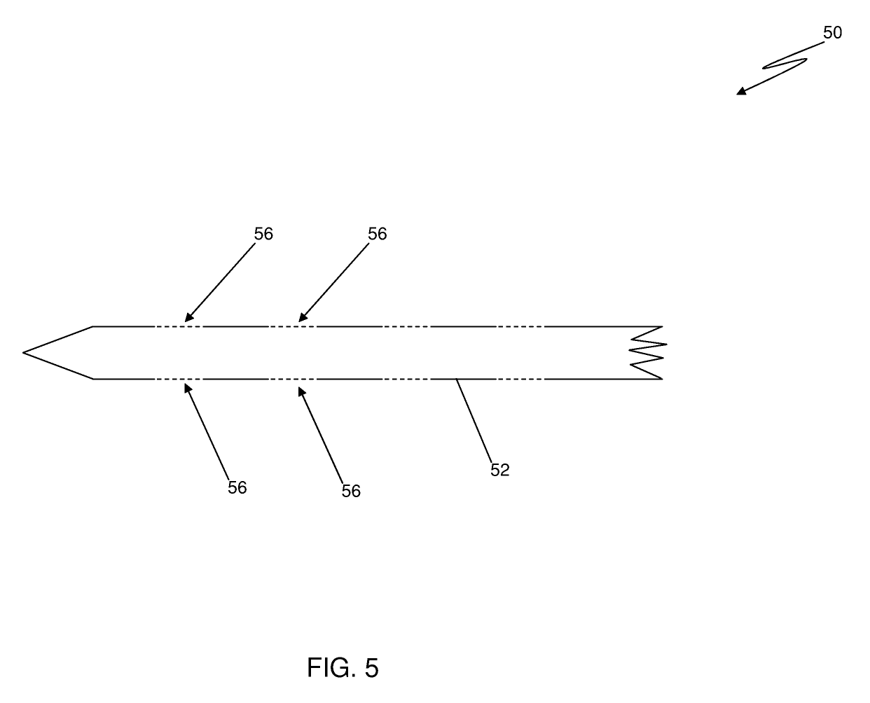

Another embodiment needle 40 is shown in FIG. 4. As shown, the needle 40 can include a plurality of small openings 46 in the sidewall of the cannula 42. For purposes of the following, “small” can be defined as 1/10 or less of the cannula 42 circumference. These openings 46 can begin, for example, 2-10 mm from the tip 44 and extend proximally (i.e, away from tip 44) for 0.1-2.5 cm along the axial length of the cannula 42 at the same angular position. Each opening 46 can be axially separated from its nearest neighbor by, for example, 0.1-1.0 cm. In yet another embodiment, the openings can be disposed at different angular positions along the axial length of the cannula as well as at different axial positions. For example, as shown by the embodiment 50 in FIG. 5, lines of small openings 56, separated from each other by 180° , can run along respective predetermined axial lengths of the cannula 52. The preferred design is two rows of relatively larger holes (i.e., 1/10^{th} or more of the circumference of the cannula 42) that are oriented within the fracture line to inject the therapeutic agent uniformly within the fracture gap.

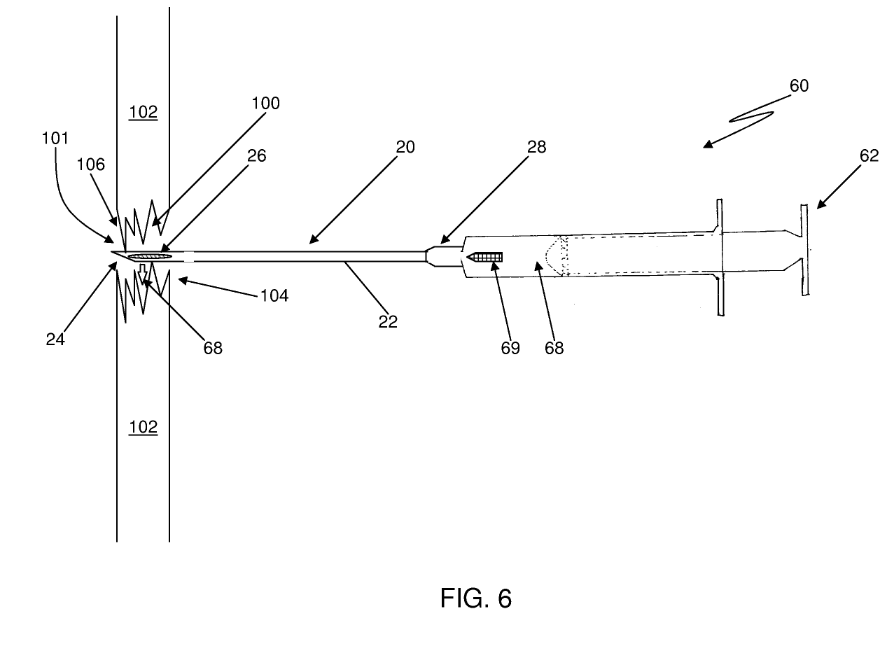

Use of a syringe 60 employing the embodiment needle 20 is shown in FIG. 6. The syringe 60 may be conventional in nature, and the proximal end of the needle 20 may include a Luer-lock 28 or any other suitable locking mechanism to attach the needle 20 to the syringe 60, as indicated earlier. An advantage of syringes that make use of the embodiment needles is that only one X-ray or fluoroscopic image is required to both position the needle and then deliver the therapeutic composition.

By way of example with the needle 20 shown in FIG. 2, the tip 24 is used to pierce the soft tissue adjacent to a closed fracture 100. The closed fracture 100 may be the result of the breakage of a bone 102 into a first exposed end 104 and a second exposed end 106. Using the syringe 60, the needle 20 is then advanced distally across the fracture line until the tip 24 is substantially positioned flush with the far lateral outer cortical wall surface 101 of the fractured bone, and only one X-ray or fluoroscopic image is required to ascertain the proper positioning of the needle 20.

As shown in FIG. 6, once properly positioned, the opening in the sidewall of the needle 20 is disposed within or adjacent to the fracture 100 and extends across a significant portion of the fracture line, such as at least across 25% of the fracture line. If the needle 20 is not directly disposed within the fracture line itself, then the needle 20 is preferably within 1-5 mm of the fracture line. The opening 26 is oriented to face the first exposed end 104 of the fractured bone or in the plane of the fracture 102. This may be done, for example, by rotating the syringe 60 to cause corresponding rotation of the needle 20. To facilitate such rotational orientation of the opening 26, the syringe 60 may include one or more markings 69 to indicate the angular positioning of the openings 26. Hence, the locking mechanism 28 on the needle 20 is designed so that when locked onto the syringe 60 the opening 26 is aligned with the corresponding opening indicator 69 on the syringe 60. Rotation of the syringe 60 may be performed before, during or after the lateral positioning of needle tip 24.

Once the needle 20 is laterally and rotationally positioned, the therapeutic composition 68 in the syringe 40 can be expelled, such as by depressing upon plunger 62, while keeping the needle 20 in a fixed position, both laterally and rotationally. Then, the syringe 60 may be rotated, such as by 180°, and the plunger depressed again so that ends 104, 106 or both sides of the fracture plane receive the therapeutic composition 68. Of course, if the needle has, for example, two rows of openings separated by 180° then only small, such as 20° or less, rotations of the syringe 60 may be needed to fully cover both ends 104, 106 or planes of the fracture line. The therapeutic composition 68 may include, for example, a bone growth factor to encourage healing of the fracture 100, including BMPs, GDFs, FGFs, PDGFs, statins or the like. It is not necessary to move the needle 20 laterally (i.e., proximally) since the composition 68 is delivered to the exposed ends 104, 106 of the fractured bone 102 perpendicularly to the axial direction of the shaft 22 of the needle 20. Further, because the tip 24 of the needle 20 is solid, none of the composition 68 is delivered from the needle tip 24. Hence, all of the therapeutic composition 68 is expelled from the needle 20 in a direction that is directly towards one or both of the exposed ends 104, 106 of the fracture 100. When the needle 20 is disposed directly within the fracture 100, the therapeutic composition 68 can be expelled substantially parallel to the longitudinal length of the fractured bone 102 and in a dispersal zone that substantially covers the entire fracture line. Alternatively the therapeutic composition 68 can be delivered perpendicular to the longitudinal length of the fractured bone 102 to fill in any empty spaces or gaps within the fracture line. Because it is not necessary to reposition the needle 20 laterally while delivering the composition 68, it is thus also not necessary to perform additional X-rays or fluoroscopic imaging.

Although discussed above in the context of closed fractures, it will be appreciated that embodiment needles could also be used in open surgical procedures as well when it is desired to deliver therapeutic compositions perpendicular to the needle axis.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

What is claimed is:

1. A needle for delivering a therapeutic composition, the needle comprising:
a cannula defining a hollow body along an axial axis of the cannula, the cannula having a proximal end and a distal end;
a tissue-piercing tip disposed at the distal end of the cannula and fluidically sealed from the hollow body; and
at least one opening in a sidewall of the cannula, the at least one opening fluidically coupled to the hollow body.

2. The needle of claim 2 further comprising at least two openings respectively disposed in opposing sidewalls of the cannula.

3. The needle of claim 1 wherein an axial extent of the at least one opening covers at least 10% of a bone fracture line.

4. The needle of claim 3 further comprising a plurality of openings that collectively extend across at least 25% of the bone fracture line.

5. The needle of claim 1 wherein the at least one opening is at least 2 mm from the tissue-piercing tip and extends proximally at least 1 mm along the axial axis of the cannula.

6. A method for treating a fracture comprising:
advancing the tip of the needle of claim 1 through soft tissue so that the at least one opening is at least partially disposed within or adjacent to the fracture;
orienting the needle so that the at least one opening faces an exposed end of the fracture or is within a plane of the fracture line; and
causing a therapeutic composition to travel through the hollow body and exit through the at least one opening.

7. The method of claim 6 wherein the fracture is a closed fracture.

8. The method of claim 6 wherein the therapeutic composition comprises a bone growth factor.

9. The method of claim 6 further comprising rotating the needle so that the at least one opening faces another exposed end of the fracture.
